(19) United States
(12) Patent Application Publication
(10) Pub. No.: US 2026/0355073 A1
(43) Pub. Date: Nov. 12, 2026

(54) AEROSOL DEVICE BASED ON AN ANIONIC FIXING POLYMER, AN IONICDIRECT DYE, A MONOALCOHOL AND A HALOGENATED HYDROCARBON

(71) Applicant: L'OREAL, paris (FR)

(72) Inventors: Carine AIRES, Saint-Ouen (FR); Liobel AUBERT, Saint-Ouen (FR); Nadia SMAIL, Saint-Ouen (FR)

(73) Assignee: L'OREAL, paris (FR)

(21) Appl. No.: 19/123,292

(22) PCT Filed: Oct. 27, 2023
(86) PCT No.: PCT/EP2023/080152

§ 371 Date: Apr. 22, 2025

(30) Foreign Application Priority Data
Oct. 28, 2022 (FR) FR2211269

(51) Int. Cl. A61K 8/04; A45D 19/00; A45D 19/02; A61K 8/31; A61K 8/34; A61K 8/81; A61Q 5/06; B65D 83/14

(52) U.S. Cl. CPC A61K 8/046; A45D 19/0066; A45D 19/02; A61K 8/315; A61K 8/34; A61K 8/8152; A61Q 5/065; B65D 83/14; A61K 2800/432; A61K 2800/87

(57) ABSTRACT

An aerosol device comprises at least one pressurized container that comprises at least one composition that comprises at least one anionic fixing polymer, at least one ionic direct dye, and at least one C₂-C₄ monoalcohol, and at least one propellant chosen from halogenated hydrocarbons. The device also comprises a means for delivering the composition. A process for neutralizing yellow and/or orangey tints of keratin fibers which have been previously bleached and/or are natural is provided employing the aerosol device and its contained composition and propellant.

### RELATED APPLICATIONS

This application is a National Phase Application which claims the benefit of and priority to International Application No. PCT/EP2023/080152 filed Oct. 27, 2023 which claims priority to French Application No. 2211269 filed on Oct. 28, 2022, which are hereby incorporated by reference in their entireties.

The present invention relates to an aerosol device comprising a composition for treating keratin fibers such as the hair, comprising at least one anionic fixing polymer, at least one ionic direct dye, at least one monoalcohol and at least one halogenated hydrocarbon propellant, and also a treatment process using said composition.

### TECHNICAL FIELD

It is well known that the hair is sensitized or weakened to varying degrees by the action of atmospheric agents and in particular light, and certain cosmetic treatments such as shampoo washing.

It is also known in particular that light has a tendency to attack the natural color of hair and also the artificial color of dyed or bleached hair. The color of the hair gradually fades or turns towards unaesthetic or undesirable shades.

Indeed, bleached hair is very often weakened and porous, so that over time it tends to lose its pigmentation and to exhibit unsightly yellow/orangey tints. Similarly, light-colored hair, in particular white or grey hair, may have unsightly yellow/orangey tints.

Thus, the aim of the present invention is to develop a device for treating keratin fibers such as the hair, allowing rapid and homogeneous application of the composition to keratin fibers leading to neutralization of the yellow and/or orangey tints of the hair over the whole of the head of hair treated, having good cosmetic properties in terms of softness or manageability of the hair and also good properties of shaping the head of hair and/or form retention of the hairstyle. Moreover, the device according to the invention results in a treatment which does not transfer or transfers little to the skin or to textiles once dried.

### DISCLOSURE OF THE INVENTION

This aim is achieved with the present invention, a subject of which is an aerosol device comprising:
at least one pressurized container comprising:
   a) at least one composition comprising:
   i) at least one anionic fixing polymer,
   ii) at least one direct dye, and
   iii) at least one C₂-C₄ monoalcohol, and
   b) at least one propellant chosen from halogenated hydrocarbons, and
a means for delivering the composition.

A subject of the invention is also a process for neutralizing the yellow and/or orangey tints of keratin fibers such as the hair, which have been previously bleached and/or are natural, comprising the application to said fibers of a composition delivered from a device as defined above.

By using the aerosol device according to the invention, a uniform and chromatic neutralization is obtained quickly and easily on the keratin fibers, such as the hair, on the whole of the head of hair. In addition, the hair is softer to the touch, more manageable. This treatment has the advantage of being a leave on treatment and of not using water.

Furthermore, this composition has good transfer resistance and the deposition onto supports with which the composition comes into contact, such as the skin and/or clothing, is limited.

The present invention also relates to a process for hair shaping and/or for form retention of the hairstyle, comprising a step of applying the composition to the hair, while dry or wet, said composition being a leave on composition. Preferably, the composition is applied to keratin fibers such as light-colored (blond or white) hair which is natural or bleached.

Another subject of the invention is the use of the composition for hair shaping and/or form retention of a hairstyle.

The term “neutralization of yellow and/or orangey tints” is intended to mean masking unsightly yellow and/or orangey tints, that is to say modifying the color to make it more natural (close to a natural blond or white), in particular by varying the values of the parameters a* (red green axis) and/or b* (yellow blue axis) of the CIELab colorimetric system.

The invention is not limited to the illustrated examples. The features of the various examples may particularly be combined within variants which are not illustrated.

The term “at least one” means one or more.

The aerosol device comprises at least one pressurized container comprising:
a) at least one composition comprising:
   i) at least one anionic fixing polymer,
   ii) at least one ionic direct dye, and
   iii) at least one C₂-C₄ monoalcohol,
b) at least one propellant chosen from halogenated hydrocarbons.

The composition a) according to the invention is preferably a cosmetic composition for treating keratin fibers, such as the hair.

In what follows, when the propellant(s) are present in the composition a), the total weight of the composition corresponds to the total weight of the composition a). When the propellant(s) are not present in the composition a), the total weight of the composition corresponds to the weight of the composition a)+the weight of the propellant(s).

### Anionic Fixing Polymer:

The composition according to the invention comprises at least one anionic fixing polymer.

The term “fixing polymer” refers to any polymer that is capable of giving a shape to a head of hair or of maintaining a head of hair in a given shape.

Anionic fixing polymers that may be mentioned include polymers including groups derived from carboxylic, sulfonic or phosphoric acids, and having a number-average molecular mass of between 500 and 5 000 000.

The carboxylic groups are provided by unsaturated monocarboxylic or dicarboxylic acid monomers, such as those corresponding to formula (I):

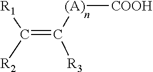

wherein
n is an integer from 0 to 10,
A denotes a methylene group, optionally connected to the carbon atom of the unsaturated group or to the adjacent methylene group, when n is greater than 1, via a heteroatom, such as oxygen or sulfur,
R₁ denotes a hydrogen atom or a phenyl or benzyl group,
R₂ denotes a hydrogen atom, an alkyl group including from 1 to 4 carbon atoms or a carboxyl group,
R₃ denotes a hydrogen atom, an alkyl group including from 1 to 4 carbon atoms, a —CH₂—COOH) group, a phenyl group or a benzyl group.

In formula (I) above, the alkyl group containing from 1 to 4 carbon atoms may in particular denote methyl and ethyl groups.

The anionic fixing polymers bearing carboxylic or sulfonic groups that are preferred are:
A) copolymers of acrylic or methacrylic acid or salts thereof, including copolymers of acrylic acid and acrylamide, and methacrylic acid/acrylic acid/ethyl acrylate/methyl methacrylate copolymers, in particular Amerhold DR 25 sold by Amerchol, and sodium salts of polyhydroxycarboxylic acids. Mention may also be made of methacrylic acid/ethyl acrylate copolymers, notably in aqueous dispersion, such as Luviflex Soft and Luvimer MAE, which are sold by BASF.
B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol and optionally crosslinked. Such polymers are described in particular in French patent 1 222 944 and German patent application No. 2 330 956, the copolymers of this type including an optionally N-alkylated and/or hydroxyalkylated acrylamide unit in their chain as described notably in Luxembourg patent applications 75370 and 75371. Mention may also be made of copolymers of acrylic acid and of C₁-C₄ alkyl methacrylate.

As another anionic fixing polymer from this class, mention may also be made of the butyl acrylate/acrylic acid/methacrylic acid branched block anionic polymer sold under the name Fixate G-100 L by Lubrizol (INCI name AMP-Acrylates/Allyl Methacrylate Copolymer).
C) crotonic acid-based copolymers, such as those including vinyl acetate or propionate units in their chain and optionally other monomers such as allylic esters or methallylic esters, vinyl ether or vinyl ester of a linear or branched saturated carboxylic acid with a long hydrocarbon-based chain, such as those including at least 5 carbon atoms, these polymers possibly being grafted and crosslinked, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products that fall within this category are the resins 282930, 261314 and 281310 sold by National Starch.

Mention may also be made, as copolymer derived from crotonic acid, of crotonic acid/vinyl acetate/vinyl tert-butylbenzoate terpolymers, and in particular Mexomere PW supplied by Chimex.
D) polymers derived from maleic, fumaric or itaconic acids or anhydrides with vinyl esters, vinyl ethers, vinyl halides, phenylvinyl derivatives, acrylic acid and its esters; these polymers may be esterified. Such polymers are described in particular in U.S. Pat. Nos. 2,047,398, 2,723,248 and 2,102,113 and GB patent 839 805, and notably those sold under the names Gantrez™ AN or ES by ISP.

Polymers also falling within this category are the copolymers of maleic, citraconic or itaconic anhydrides and of an allylic or methallylic ester optionally including an acrylamide or methacrylamide group, an α-olefin, acrylic or methacrylic esters, acrylic or methacrylic acids or vinylpyrrolidone in their chain, the anhydride functions being monoesterified or monoamidated. These polymers are described, for example, in French patents 2 350 384 and 2 357 241 by the applicant.
E) polyacrylamides including carboxylate groups.
F) polymers comprising sulfonic groups. These polymers may be polymers including vinylsulfonic, styrenesulfonic, naphthalenesulfonic, acrylamidoalkylsulfonic or sulfoisophthalate units.

These polymers may notably be chosen from:
polyvinylsulfonic acid salts with a molecular mass of between 1000 and 100 000 approximately, and also the copolymers with an unsaturated comonomer such as acrylic or methacrylic acids and esters thereof, and also acrylamide or derivatives thereof, vinyl ethers and vinylpyrrolidone;
polystyrenesulfonic acid salts and sodium salts, with a molecular mass of about 500 000 and of about 100 000. These compounds are described in patent FR 2 198 719;
polyacrylamidesulfonic acid salts such as those mentioned in patent U.S. Pat. No. 4,128,631;
G) grafted anionic silicone polymers.

The grafted silicone polymers used are preferentially chosen from polymers bearing a non-silicone organic backbone grafted with monomers containing a polysiloxane, polymers bearing a polysiloxane backbone grafted with non-silicone organic monomers, and mixtures thereof.
H) anionic polyurethanes, which may include silicone grafts and silicones bearing hydrocarbon-based grafts.

Examples of fixing polyurethanes that may notably be mentioned include the dimethylolpropionic acid/isophorone diisocyanate/neopentyl glycol/polyester diols copolymer (also known under the name polyurethane-1, INCI name) sold under the brand name Luviset™ PUR by BASF, and the dimethylolpropionic acid/isophorone diisocyanate/neopentyl glycol/polyester diols/silicone diamine copolymer (also known under the name polyurethane-6, INCI name) sold under the brand name Luviset™ Si PUR A by BASF.

Another anionic polyurethane that may also be used is Avalure UR 450.

Polymers containing sulfoisophthalate groups, such as the polymers AQ55 and AQ48 sold by Eastman, may also be used.

According to the invention, the anionic fixing polymers are preferably chosen from acrylic acid copolymers such as the acrylic acid/ethyl acrylate/N-tert-butylacrylamide terpolymer sold under the name Ultrahold Strong™ by BASF, methacrylic acid/ethyl acrylate copolymers, in particular in aqueous dispersion, such as Luviflex Soft and Luvimer MAE sold by BASF, copolymers derived from crotonic acid such as vinyl acetate/vinyl tert-butylbenzoate/crotonic acid terpolymers and crotonic acid/vinyl acetate/vinyl neododecanoate terpolymers sold under the name Resin 28-29-30 by National Starch, polymers derived from maleic, fumaric or itaconic acids or anhydrides with vinyl esters, vinyl ethers, vinyl halides, phenylvinyl derivatives, acrylic acid and its esters, such as the monoesterified methylvinylether/maleic anhydride copolymer sold under the name Gantrez™ ES 425 by ISP, Luviset SI PUR, Mexomere PW, anionic polyurethanes, elastomeric or otherwise, polymers containing sulfoisophthalate groups, anionic fixing polymers of family B), and even more particularly, crotonic acid/vinyl acetate/vinyl neododecanoate terpolymers sold under the name Resin 28-29-30 by National Starch, are preferably used.

The total amount of fixing polymer(s) may range from 0.1% to 30% by weight relative to the weight of the composition, preferably from 1% to 10% by weight relative to the weight of the composition, preferably from 2% to 8% by weight, better still from 3% to 7% by weight relative to the weight of the composition.

### Ionic Direct Dye:

The composition according to the invention comprises at least one ionic direct dye. Preferably, the ionic direct dye(s) according to the invention are chosen from blue or violet dyes.

The ionic direct dyes that can be used according to the invention are preferably anionic or cationic and preferably anionic direct dyes.

The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from acid azo direct dyes, acid azine dyes, acid triarylmethane dyes, acid indoamine dyes, acid anthraquinone dyes, xanthene dyes, indigoids and acid natural dyes.

As anionic azo dyes, mention may be made of the dyes Acid Violet 7, Acid Violet 14, Acid Blue 113 and Acid Blue 117.

As anionic acid anthraquinone dyes, mention may be made of the dyes Acid Blue 25, Acid Blue 43, Acid Blue 62, Acid Blue 78, Acid Blue 129, Acid Blue 138, Acid Blue 140, Acid Blue 251, Acid Violet 42, Acid Violet 43 (also called EXT violet No. 2).

As anionic examples of triarylmethane dyes, mention may be made of the dyes Acid Blue 1; Acid Blue 3; Acid Blue 7; Acid Blue 9; Acid Violet 49.

As examples of anionic xanthene dyes, mention may be made of: Acid Violet 9.

As anionic examples of indole dyes, mention may be made of: Acid Blue 74.

As cationic direct dyes which can be used according to the invention, mention may be made of the dyes Basic Blue 6, Basic Blue 7, Basic Blue 9, Basic Blue 26, Basic Blue 41, Basic Blue 99, Basic Violet 1, Basic Violet 2, Basic Violet 3, Basic Violet 10, Basic Violet 14, and Basic Yellow 87.

The preferred direct dyes are Ext. Violet 2, Acid Blue 62, Acid Blue 9 and Basic Violet 2, more preferentially Ext. Violet 2, Acid Blue 62 and Acid Blue 9.

The direct dye(s) is (are) present in a total amount which may range from 0.001% to 3%, preferably from 0.005% to 1%, better still from 0.0075% to 0.5% by weight relative to the total weight of the composition. Preferably the direct dyes are ionic dyes,

Preferably, the anionic direct dye(s) is (are) present in a total amount which may range from 0.001% to 3%, preferably from 0.005% to 1%, better still from 0.0075% to 0.5% by weight relative to the total weight of the composition.

C₂-C₄ Monoalcohols

The composition according to the present invention comprises one or more C₂-C₄ monoalcohols.

The C₂-C₄ monoalcohols are notably chosen from ethanol, isopropanol, tert-butanol and n-butanol. Preferably, the monoalcohol used is ethanol.

The C₂-C₄ monoalcohols are generally present in a total amount ranging from 10% to 80% by weight relative to the total weight of the composition, and more preferentially ranging from 20% to 70% by weight and even more particularly ranging from 30% to 60% by weight relative to the total weight of the composition.

### Additives:

The compositions may also comprise at least one agent commonly used in cosmetics, for example chosen from reducing agents, organic solvents other than C₂-C₄ monoalcohols, softeners, antifoams, moisturizers, UV-screening agents, peptizers, solubilizers, fragrances, anionic, cationic, nonionic or amphoteric surfactants, proteins, vitamins, silicones, mineral oils, vegetable oils, fatty esters, fatty ethers and pH modifiers, and mixtures thereof.

### Organic Solvents:

The composition according to the invention may comprise one or more organic solvents other than C₂-C₄ monoalcohols.

Mention may be made, as organic solvent, of polyols and polyol ethers such as pentylene glycol, hexylene glycol, caprylyl glycol, 2-butoxyethanol, propylene glycol, dipripropylene glycol, propylene glycol monomethyl ether, diethylene glycol monoethyl ether and monomethyl ether, and also aromatic alcohols such as benzyl alcohol or phenoxyethanol, and mixtures thereof.

When they are present, the organic solvents are present in a total content preferably between 1% and 80% by weight approximately relative to the total weight of the composition and more preferentially between 10% and 70% by weight approximately and even more particularly between 20% and 60% by weight relative to the total weight of the composition.

The composition according to the invention may comprise water which may preferably be present in a content of less than 10% by weight, more preferentially less than 5% by weight relative to the weight of the composition.

More preferentially, the composition does not include water added during the preparation of the composition. The water possibly present may correspond to the residual water provided by the mixed ingredients.

Preferably, the composition according to the invention is anhydrous (0% water).

### Device:

The aerosol device comprises at least one pressurized container comprising at least one propellant b).

The propellant(s) b) may optionally be present in the composition a) as previously described.

The container of the device according to the invention can have rigid walls and can directly contain the composition a).

As a variant, the container can have rigid walls and can contain a flexible-walled bag which contains the composition a).

According to this configuration, the composition a) in the bag may not comprise propellant b), the latter being in the volume defined between the rigid walls of the container and the bag.

Preferably, the composition a) contained in the bag itself also comprises at least one propellant b).

Preferably, the composition a) comprises one or more propellants b), and is directly contained in the container of the device.

The device comprises a means for delivering the compositions, the means comprising at least one dispensing valve which surmounts the container.

The valve is in selective fluidic communication with the inside of the container via a valve inlet orifice, the communication being established in response to the activating of an activation means, such as a push-button.

When the device comprises a rigid-walled container which contains a flexible bag, the valve is then equipped with two inlet orifices, one of the orifices being able to communicate with the inside of the bag and the other being able to communicate with the volume defined between the bag and the rigid walls of the container.

When the container does not contain a bag, it is equipped with a dip tube which makes it possible to convey the composition to the inlet orifice of the dispensing valve.

When the container contains a bag, the inlet orifice of the valve opens into the bag.

The device can comprise at least one diffuser which caps the valve. The push-button may form part of the diffuser.

The diffuser may be equipped with one or more dispensing pipe(s) provided to convey the composition(s) up to one or more dispensing orifices.

Documents U.S. Pat. Nos. 3,917,121, 4,720,046 and WO 00/76880 disclose examples of devices.

The diffuser may comprise a single outlet orifice and diffusion branches that radiate from said orifice. As a variant, the diffuser comprises a plurality of outlet orifices. Advantageously, the outlet orifices may be arranged so as to obtain a diffusion grille.

Preferably, the pressurized container of the device according to the invention has rigid walls and directly contains the composition a).

Preferably, the composition a) comprises one or more propellants b).

The propellant(s) b) are present in the pressurized container according to the invention preferably in a total content ranging from 25% to 80% by weight, more preferably from 30% to 70% by weight, better still from 35% to 60% by weight, even better still from 40% to 50% by weight relative to the total weight of the composition (packaged in the aerosol device). When the propellant(s) are present in the composition a), the total weight of the composition corresponds to the total weight of the composition a). When the propellant(s) are not present in the composition a), the total weight of the composition corresponds to the weight of the composition a)+the weight of the propellant(s).

Preferably, the propellant(s) b) are present in the pressurized container according to the invention in a total amount ranging from 25% to 80% by weight, more preferentially from 30% to 70% by weight, better still from 35% to 60% by weight, even better still from 40% to 50% by weight relative to the total weight of the composition a).

The propellant(s) b) is (are) chosen from halogenated hydrocarbons and mixtures thereof. Preferably, the propellant(s) b) is (are) chosen from C₂-C₄ halogenated hydrocarbons and mixtures thereof. Mention may be made in particular of fluorinated and/or chlorinated hydrocarbons such as, for example, 1,2-difluoroethane, chlorodifluoromethane, dichlorodifluoromethane, 1,2-difluoroethane, chlorodifluoroethane, dichlorotetrafluoroethane, trichlorofluoromethane, 1,1,1,2-tetrafluoroethane, chloropentafluoroethane, 1-chloro-1,1-difluoroethane, 1,1-difluoroethane, etc., and mixtures thereof.

More preferentially, the device according to the invention comprises 1,1-difluoroethane (HFC 152A).

When the propellant(s) are present in the composition a), the total amount of anionic fixing polymer may range from 0.1% to 30% by weight relative to the weight of the composition a), preferably from 1% to 10% by weight relative to the weight of the composition a), preferably from 2% to 8% by weight, better still from 3% to 7% by weight relative to the total weight of the composition a).

When the propellant(s) are present in the composition a), the total amount of direct dye(s) may range from 0.001% to 3%, preferably from 0,005% to 1%, better still from 0.0075% to 0.5% relative to the total weight of the composition a).

The propellants are pressurized, more particularly at least partially pressurized in liquid form.

Advantageously, the composition which is dispensed from the pressurized container is in liquid form.

### Process:

Another subject of the invention is a process for treating keratin fibers such as the hair, comprising the application to said fibers of a composition delivered from a device as defined above.

The composition delivered by the aerosol device described above may be used on dry keratin fibers, which are preferably light blond and white in color, and natural or bleached. Preferably, the keratin fibers are white or have a tone depth of greater than or equal to 7, better still greater than 8, preferentially greater than 9. The “tone” depths are based on the classification of natural shades from 1 to 10, a tone separating each shade from the one immediately following or preceding it (see “Science of hair treatments” by C. Zviak, published by Masson 1988, p. 278).

By virtue of the device according to the invention, the application is uniform on the whole of the head of hair.

The present invention will now be described more specifically by means of examples, which do not in any way limit the scope of the invention. However, the examples make it possible to support specific features, variants and preferred embodiments of the invention.

### EXAMPLES

### Example 1

The compositions A to D according to the invention were prepared.

Compositions (g/100 g) AM: Active material

TABLE 1

| | A | B | C | D |
|---|---|---|---|---|
| VA/Crotonates/Vinyl | 6 | 5.5 | 6 | 6 |
| Neodecanoate Copolymer | | | | |
| Aminomethyl Propanol | 0.62 | 0.58 | 0.62 | 0.62 |
| Ethanol | Qs 100 | Qs 100 | Qs 100 | Qs 100 |
| Water | | 3.00 | | |
| Ext. Violet 2 / CI 60730 | 0.01 | 0.01 | | |
| Acid Blue 9 / CI 42090 | | | 0.01 | |
| Basic Violet 2 / CI 80399 | | | | 0.01 |
| Hydrofluorocarbon 152A | 45 | 45 | 45 | 45 |
| Fragrance/perfume | 0.28 | 0.28 | 0.28 | 0.28 |

The compositions were packaged in a can with the following equipment:

Valve: Precision GI—Inner Nozzle—0.33/RI—Inner Restriction—0.33 stem 3.3 and a Microdiffuser with nozzle DU3832 (0.38 mm)

The aerosol device according to the invention is actuated and the mixture is delivered in spray form.

Composition A was sprayed for 5 seconds onto each of the 2 sides of locks of bleached hair (alkaline solubility 50) weighing 2.7 g, at a distance of 20 cm.

After drying, colorimetric measurements were taken using a Konica-Minolta-Post FR-C-MCI-0788 spectrocolorimeter (illuminant D65, angle 10°, specular components included) in the CIELab system.

In this system, a* represents the red-green axis, b* represents the yellow-blue axis. The chromaticity C* is calculated according to the formula:

C*=(a*²+b*²)^{1/2}

The lower the value of C*, the lower the chromaticity, and the less the yellow and/or orangey tints are visible:

TABLE 2

| | C* |
|---|---|
| Before application of composition A | 26.0 |
| After 1 application of composition A | 22.0 |

The value of C* is significantly lower after application of composition A: The color is less chromatic.

It is also observed visually that the unsightly yellow and/or orangey tints are markedly reduced and that the bleached hair has a more natural visual appearance. The distribution of the product along the locks is homogeneous. The color is therefore more uniform. In addition, the fixing is natural and allows the hairstyle to be held.

### Example 2

The following compositions were prepared (g/100 g) AM: Active material

TABLE 3

| | Ei | Ec |
|---|---|---|
| | (invention) | (comparison) |
| VA/CROTONATES/VINYL | 6 | 6 |
| NEODECANOATE COPOLYMER | | |
| AMINOMETHYL PROPANOL | 0.62 | 0.62 |
| ETHANOL | Qs 100 | Qs 100 |
| water | 3 | 3 |
| EXT. VIOLET 2 = ACID VIOLET 43 | 0.01 | — |
| DISPERSE VIOLET 1 | — | 0.01 |
| HYDROFLUOROCARBON | 45 | 45 |

The compositions were packaged in a can as disclosed in example 1

The aerosol device according to the invention is actuated and the mixture is delivered in spray form.

Compositions A was sprayed for 5 seconds onto each of the 2 sides of locks of bleached hair (alkaline solubility 50) weighing 2.7 g, at a distance of 20 cm.

After drying, colorimetric measurements were taken using a Konica-Minolta-Post FR-C-MCI-0788 spectrocolorimeter (illuminant D65, angle 10°, specular components included) in the CIELab system.

In this system, a* represents the red-green axis, b* represents the yellow-blue axis.

The lower the value of b*, the lower the yellow hue is, and the less the yellow and/or orangey tints are visible:

TABLE 4

| | b* |
|---|---|
| Before treatment | 30.2 |
| Ei invention | 24.3 |
| Ec comparatif | 28.5 |

The bleached hair treated with composition Ei according to the invention has a lower value of b* than the bleached hair treated with comparative composition Ec.

It can be concluded that the composition Ei according to the invention leads to a reduced yellowiness.

1. An aerosol device comprising:
at least one pressurized container comprising:
a) at least one composition comprising:
i) at least one anionic fixing polymer,
ii) at least one ionic direct dye, and
iii) at least one C₂-C₄ monoalcohol,
b) at least one propellant chosen from halogenated hydrocarbons,
a means for delivering the composition.

2. The device as claimed in claim 1, wherein said at least one anionic fixing polymer is chosen from acrylic acid copolymers or salts thereof, methacrylic acid copolymers or salts thereof, crotonic acid copolymers, polyacrylamides bearing carboxylate groups, homopolymers bearing sulfonic groups, copolymers bearing sulfonic groups, anionic polyurethanes, anionic grafted silicone polymers, or mixtures thereof.

3. The device as claimed in claim 1, wherein said at least one anionic fixing polymer is chosen from terpolymers of crotonic acid, terpolymers of vinyl acetate, terpolymers of vinyl neodecanoate, or mixtures thereof.

4. The device as claimed in claim 1, wherein the at least one anionic fixing polymer is present in a total amount ranging from 0.1% to 30% by weight relative to the total weight of the composition.

5. The device as claimed in claim 1, wherein the at least one ionic direct dye is chosen from anionic direct dyes or mixtures thereof.

6. The device as claimed in claim 1, wherein the at least one ionic direct dye is chosen from azo, anthraquinone, triarylmethane, xanthene dyes, or mixtures thereof.

7. The device as claimed in claim 1, wherein the at least one ionic direct dye is chosen from anionic blue, violet direct dyes, or mixtures thereof.

8. The device as claimed in claim 1, wherein the at least one ionic direct dye is present in a total amount ranging from 0.001% to 3% by weight relative to the total weight of the composition.

9. The device as claimed in claim 1, wherein the at least one C₂-C₄ monoalcohol is chosen from ethanol, isopropanol, tert-butanol and n-butanol, or mixtures thereof.

10. The device as claimed in claim 1, characterised in that the at least one C₂-C₄ monoalcohol is present in a total amount ranging from 10% to 80% by weight relative to the total weight of the composition.

11. The device as claimed in claim 1, wherein the at least one propellant is chosen from fluorinated hydrocarbons, chlorinated hydrocarbons, or mixtures thereof.

12. The device as claimed in claim 1, wherein the at least one propellant is 1,1-difluoroethane.

13. The device as claimed in claim 1, wherein said at least one propellant is present in a total amount ranging from 25% to 80% by weight relative to the total weight of the composition.

14. The device as claimed in claim 1, wherein the container has rigid walls and directly contains the composition a) comprising the propellant b).

15. A process for treating keratin fibers, comprising application to the keratin fibers of a composition delivered from a device as claimed in claim 1.

16. The process according to claim 15, wherein application to the keratin fibers is for neutralizing yellow and/or orangey tints of keratin fibers which have been previously bleached or are natural.

17. The process according to claim 15, wherein application to the keratin fibers is for shaping keratin fibers which have been previously bleached or are natural.

18. The device as claimed in claim 1, wherein said at least one anionic fixing polymer is chosen from acrylic acid copolymers or salts thereof, methacrylic acid copolymers or salts thereof, crotonic acid copolymers, or mixtures thereof, and wherein the at least one C2-C4 monoalcohol comprises ethanol, and wherein the at least one propellant is chosen from 1,2-difluoroethane, chlorodifluoromethane, dichlorodifluoromethane, chlorodifluoroethane, dichlorotetrafluoroethane, trichlorofluoromethane, 1,1,1,2-tetrafluoroethane, or mixtures thereof, and wherein the at least one anionic fixing polymer is present in a total amount ranging from 1% to 10%, and wherein the at least one ionic direct dye is present in a total amount ranging from 0.0075% to 0.5%, and wherein the at least one C2-C4 monoalcohol is present in a total amount ranging from 20% to 70%, and wherein the at least one propellant is present in a total amount ranging from 30% to 70%, all amounts by weight relative to the total weight of the composition.

19. An aerosol device-contained composition for treating keratin fibers, comprising:
at least one anionic fixing polymer chosen from acrylic acid copolymers or salts thereof, methacrylic acid copolymers or salts thereof, crotonic acid copolymers, or mixtures thereof, the at least one anionic fixing polymer present in a total amount ranging from 1% to 10%,
at least one ionic direct dye chosen from anionic blue, violet direct dyes, or mixtures thereof, the at least one ionic direct dye present in a total amount ranging from 0.0075% to 0.5%,
at least one monoalcohol present in a total amount ranging from 20% to 70% by weight, and
at least one halogenated hydrocarbon propellant present in a total amount ranging from 30% to 70% by weight,
all amounts by weight relative to the total weight of the composition.

20. The aerosol device-contained composition for treating keratin fibers according to claim 19, the at least one anionic fixing polymer comprising VA/Crotonates/Vinyl Neodecanoate Copolymer, the at least one ionic direct dye comprising Ext. Violet 2 and Acid Blue 9, the at least one monoalcohol comprising aminomethyl propanol and ethanol, and the at least one halogenated hydrocarbon propellant comprising Hydrofluorocarbon 152A.
